(19) United States
(12) Patent Application Publication
(10) Pub. No.: US 2013/0217774 A1
(43) Pub. Date: Aug. 22, 2013

(54) NOVEL PROCESS FOR PREPARING DEXIBUPROFEN READY TO COMPRESS GRANULES

(71) Applicants: Vivekanandan Sundaramoorthy, Coimbatore District (IN); Anandsenthilvel Palanisamy, Coimbatore District (IN); Sampathkumar Devarajan, Chennai (IN); Jeffrey Stuart Bergman, Hertforshire (GB)

(72) Inventors: Vivekanandan Sundaramoorthy, Coimbatore District (IN); Anandsenthilvel Palanisamy, Coimbatore District (IN); Sampathkumar Devarajan, Chennai (IN); Jeffrey Stuart Bergman, Hertforshire (GB)

(21) Appl. No.: 13/881,186

(22) PCT Filed: Oct. 24, 2011
(86) PCT No.: PCT/IN11/00735

§ 371 Date: Apr. 24, 2013

(30) Foreign Application Priority Data
Oct. 24, 2010 (IN) 2437/CHE/2010

(51) Int. Cl. A61J 3/00; A61K 31/192

(52) U.S. Cl. CPC A61J 3/00; A61K 31/192
USPC 514/570; 264/115

(57) ABSTRACT

Preparing a dexibuprofen pharmaceutical formulation in the form of granules that can be directly compressed into tablets. The process consists of two steps: (i) preparation of base granules, and (ii) blending base granules with a compression aid. The process of preparing base granules involves use of liquid bed granulator with top spray method, or slugging by roll compaction. The granules prepared by using both the methods showed improved compressibility and flowability compared to granules prepared by conventional granulation process using rapid mixer granulator. The ready-to-compress granules prepared by the process show no signs of sticking when compressed on a high-speed commercial scale tablet compression machine.

### TECHNICAL FIELD OF THE INVENTION

The present invention relates to novel process of preparing both dexibuprofen and its pharmaceutical formulation in the form of ready to compress granules to aid in free flow, which can be directly compressed into tablets.

### BACKGROUND OF THE INVENTION

Ibuprofen [R,S-(±)-2-(4-isobutylphenyl)-propionic acid] has one chiral center, thus there are two enantiomers, S(+)-ibuprofen and R(−)-ibuprofen, the S(+)-ibuprofen is also known as dexibuprofen. Ibuprofen is a poorly water-soluble drug with a melting point of 75° C. to 77.5° C., while both enantiomers of ibuprofen, S(+)-ibuprofen and R(−)-ibuprofen, melt at 52° C. to 54° C.

Ibuprofen has proved to be difficult to formulate in the form of tablets, and this is believed to be due to its poor compressibility and flowability. The poor compressibility and flowability is believed to be partly due to the fact that ibuprofen itself has a very low bulk density, and partly due to the low melting point.

In the case of dexibuprofen, the above-mentioned problems of poor compressibility and flowability associated with the preparation of tablets containing ibuprofen are considerably more pronounced, owing to the fact that the melting point of dexibuprofen is significantly lower than for the ibuprofen. Furthermore, dexibuprofen has a very low bulk density. The low melting point also causes frequent sticking problem during'compression on high speed commercial scale tablet compression machine.

Following is given a brief review of patent literature relating to dexibuprofen formulation and profess of preparing the same:

U.S. Pat. No. 4,877,620 discloses a pharmaceutical tablet compositions comprising 62.5% w/w S-(+)-ibuprofen, 4.2% w/w insoluble polyvinylpyrrolidone, 31.3% w/w microcrystalline cellulose and 2% w/w magnesium stearate, process of preparing the same is not disclosed.

U.S. Pat. No. 5,869,102 discloses a solid pharmaceutical composition containing dexibuprofen in the form of tablet comprising of dexibuprofen, microcrystalline cellulose (30-50% w/w), colloidal silica (less than 0.3% w/w) and wetting agent (3-6% w/w) or surfactant (0.01-2% w/w of sodium lauryl sulfate, Tween, Span), wherein the tablets are prepared by direct compression.

PCT application no WO199410993 discloses a pharmaceutical particulate formulation comprising dexibuprofen, and a water-soluble binder, wherein the particulate formulation comprise of a blend containing i) granulated material incorporating the active drug substance and ii) extragranular excipients in powder form and the granules are prepared by conventional wet granulation process using granulator.

PCT application no WO2009084041 discloses a pharmaceutical composition comprising of dexibuprofen or its pharmaceutically acceptable salt and process of preparing the same, having dexibuprofen mean particle size equal to or less than 125 microns with in-vitro dissolution of more than about 90% in 5 minutes, wherein the preferred process involves wet granulation using high shear granulator.

PCT application no WO1994017793 discloses a process for the preparation of a tablet containing dexibuprofen which rapidly releases the active compound, wherein the tablet consist of dry binder intra and extra granularly and prepared by dry granulation process.

U.S. Pat. No. 5,560,926 discloses a process for the production of a tablet containing S-ibuprofen which rapidly releases the active compound, and to S-ibuprofen-containing tablets prepared by this process.

The prior art discloses various process of preparing solid pharmaceutical formulation containing dexibuprofen including direct compression, dry granulation and wet granulation using conventional granulator to overcome the formulation difficulties associated with dexibuprofen due to its low melting point, low density and poor flowability.

Thus there is constant need of developing a robust process of preparing dexibuprofen pharmaceutical formulation in the form of granules which can be directly compressed into tablets and at the same time should also overcome the formulation difficulties associated with dexibuprofen.

### SUMMARY OF THE INVENTION

The present invention relates to a novel process of preparing both dexibuprofen and its pharmaceutical formulation in the form of granules, which can be directly compressed into tablets. It was surprisingly found that the dexibuprofen granules prepared by slugging (by roll compactor) and pharmaceutical formulation containing dexibuprofen along with other pharmaceutical excipients(s)/additives in the form of granules using fluid bed granulation process showed improved compressibility and flowability compared to granules prepared by conventional granulation process using rapid mixer granulator.

The dexibuprofen granules prepared using slugging process and pharmaceutical formulation containing dexibuprofen along with few additives in the form of granules prepared using fluid bed granulation process showed no signs of sticking when compressed on high speed commercial scale tablet compression machine, whereas the granules prepared by conventional granulation process using rapid mixer granulator showed signs of sticking from the beginning of compression.

The compressed tablets of granules prepared by using fluid bed granulation process showed no signs of tablet shape deformation when subjected to the stress stability study at 50° C./90% RH for one month.

### DETAILED DESCRIPTION

The present invention relates to novel process of preparing both dexibuprofen and its pharmaceutical formulation containing dexibuprofen along with few additives in the form of granules, where the former will be used as ready to compress granules and the later, which can be directly compressed into tablets, more specifically relates to a novel process of preparing dexibuprofen and its pharmaceutical formulation as ready-to-compress granules.

One of the objectives of the present invention is to prepare dexibuprofen granules, which can be directly compressed into tablets, wherein the granules comprise of mixture or blend of base granules and compression aid.

Another objective of the present invention is to prepare dexibuprofen ready to compress granules, which can be directly compressed into tablets, wherein the granules comprise of mixture or blend of base granules and compression aid.

Another objective of the present invention is to prepare dexibuprofen granules, which can be directly compressed into tablets, wherein (a) the granules comprise of mixture or blend of base granules and compression aid, and (b) the base granules were prepared by fluid bed granulation process.

Another objective of the present invention is to prepare dexibuprofen granules, which can be directly compressed into tablets, wherein, (a) the granules comprise of mixture or blend of base granules and compression aid and (b) the base granules were prepared by wet granulation process using fluid bed granulator with bottom spray method.

Yet another objective of the present invention is to prepare dexibuprofen granules, which can be directly compressed into tablets, wherein, (a) the granules comprises of a mixture or blend of base granules and compression aid and (b) the base granules were prepared by wet granulation process using fluid bed granulator with top spray method.

According to the present invention the novel process of preparing dexibuprofen granules which can be directly compressed into tablets mainly comprise of two steps: (a) preparation of base granule, and (b) blending of base granules with compression aid, wherein the process of preparing base granules, comprising the steps of: (a) mixing or blending of sifted dexibuprofen, and other pharmaceutically acceptable excipient(s), in fluid bed granulator, (b) granulation of fluidized dry mix obtained in step (a) using binder solution with top spray method to get wet granules, (c) drying of fluidized wet granules at suitable product bed temperature to obtain granules with loss on drying of not more than 0.3% w/w at 50° C., (d) sizing the dried granules of step (c) using suitable method to get base granules, and (e) the blending of base granules with compression aid involves mixing or blinding of base granules with previously sifted super disintegrant(s), glidant(s), lubricant(s) and diluent(s).

The other objectives of the present invention is to prepare dexibuprofen granules, which can be directly compressed into tablets,

Another objective of the present invention is to prepare dexibuprofen ready to compress granules, which can be directly compressed into tablets, along with other pharmaceutical additives and compression aid.

Another objective of the present invention is to prepare dexibuprofen ready to compress granules s, which can be directly compressed into tablets, wherein (a) the granules comprise of dexibuprofen and compression aid, and (b) the base granules were prepared by dry process.

Another objective of the present invention is to prepare dexibuprofen granules, which can be directly compressed into tablets, wherein, (a) the granules comprise of dexibuprofen and compression aid and (b) the base granules were prepared by slugging process using Roll compactor comprising augur assembly.

Yet another objective of the present invention is to prepare dexibuprofen granules in the form of extrudes, which can be directly compressed into tablets, wherein, (a) the granules (extrudes) comprises of dexibuprofen and compression aid and (b) the base granules were prepared by hot melt extrusion process using extruder assembly.

According to the present invention the novel process of preparing dexibuprofen granules which can be directly compressed into tablets mainly comprise of two steps: (a) preparation of base granule, and (b) blending of base granules with compression aid, wherein the process of preparing base granules, comprising the steps of: (a) Slugging dexibuprofen through Roll compactor (b) Passing the obtained slugs through the multi mill fitted, with 3.5 mm screen (c) The 3.5 mm screened granules were passed through #24 mesh and the retains passed through multi mill fitted with 1.5 mm screen (d) The 1.5 mm screened granules were again passed through #24 mesh. The #24 mesh passed granules of step c & d were passed through #40 mesh (e) the #24 mesh retains of step c & #40 mesh passed granules of step d were passed through roll compactor, and (f) the slugs obtained in step-e were repeated as per the steps from b to e. until a desired yield of #40 mesh retained granules are obtained (g) the blending of base granules with compression aid involves mixing or blinding of obtained base granules (f) with previously sifted super disintegrant(s), glidant(s), lubricant(s) and diluent(s).

According to the present invention compression aid may be comprise of one or more selected from the group of lubricant(s), glidant(s), super disintegrant(s), and diluent(s).

According to the present invention other pharmaceutically acceptable excipient(s) may be comprise of one or more selected from the group of glidant(s), super disintegrant(s), surfactant(s) and diluent(s).

According to the present invention binder solution may be aqueous based or, hydroalcoholic based or alcoholic based, preferably aqueous based.

According to the present invention sizing of dried granules can be done using suitable size sieve and/or multi mill or cone mill.

According to the present invention base granules can be prepared by using fluid bed granulation process with top spray or bottom spray method, preferably with top spray method.

According to the present invention base granules or extrudes can be prepared by using dry granulation process which involves slugging by roll compactor and or by hot melt extrusion process which involves extruders, but preferably by roll compactor method.

According to the present invention one or more diluent(s) can be selected from the group comprising of saccharides like lactose, sucrose, dextrose, sugar alcohols like mannitol, xylitol, sorbitol, starch, silicates like magnesium silicate, aluminum silicate, microcrystalline cellulose, polyethylene glycols solid grades (PEG average molecular weight greater than 1000) like PEG 1000, PEG 2000, PEG 6000, dibasic calcium phosphate dihydrate, pregelatinized starch, calcium carbonate, calcium sulfate, dibasic calcium phosphate dihydrate, tribasic calcium phosphate, kaolin, magnesium carbonate, magnesium oxide, maltodextrin and the like.

According to the present invention one or more binder(s) can be selected from the group comprising of povidone, polyvinyl alcohol, starch, pregelatinized starch, gum acacia, gum tragacanth, guar gum, pectin, copolyvidone, gelatin, sodium alginate, polymethacrylates, carbomer, saccharides like lactose, sucrose, dextrose, sugar alcohols like mannitol, xylitol, sorbitol, and the like.

According to the present invention one or more super disintegrant(s) can be selected from the group comprising of crospovidone, sodium starch glycolate, croscarmellose sodium, calcium carboxymethylcellulose, starch and the like.

According to the present invention one or more lubricant(s) and/or glidant(s) can be selected from the group comprising of magnesium stearate, stearic acid, calcium stearate, zinc stearate, talc, carnauba wax, hydrogenated vegetable oils, mineral oil, polyethylene glycols, glyceryl behenate, sodium lauryl sulfate, magnesium lauryl sulfate, sodium stearyl fumarate, colloidal silicon dioxide, and the like.

According to the present invention optionally one or more surfactant(s) can be selected from the group comprising of sodium alkyl sulfates such as sodium lauryl sulfate and sodium myristyl sulfate, sodium N-acyl sarcosinates such as sodium N-lauroyl sarcosinate and sodium N-myristoyl sarcosinate, sodium dodecylbenzenesulfonate, sodium hydrogenated coconut fatty acid monoglyceride sulfate, sodium lauryl sulfoacetate, N-acyl glutamates such as N-palmitoyl glutamate, N-methylacyltaurin sodium salt, N-methylacylalanine sodium salt, sodium a-olefin sulfonate, sodium dioctylsulfosuccinate; N-alkylaminoglycerols such as N-lauryldiaminoethylglycerol and N-myristyldiaminoethylglycerol, N-alkyl-N-carboxymethylammonium betaine, sodium 2-alkyl-1-hydroxyethylimidazoline betaine; polyoxyethylenealkyl ether, polyoxyethylenealkylaryl ether, polyoxyethylenelanolin alcohol, polyoxyethyleneglyceryl monoaliphatic acid ester, polyoxyethylenesorbitol aliphatic acid ester, polyoxyethylene aliphatic acid ester, higher aliphatic acid glycerol ester, sorbitan aliphatic acid ester, pluronic type surface active agent, and polyoxyethylenesorbitan aliphatic acid esters such as polyoxyethylenesorbitan monooleate and polyoxyethylenesorbitan monolaurate and the like.

According to the present invention general composition and process of preparing dexibuprofen ready to compress granules is as follows:

TABLE 1
General composition of dexibuprofen ready to
compress granules is as follows:

| | | |
|---|---|---|
| Sr. No. | Ingredients | % by weight |

Base granules

| | | |
|---|---|---|
| 1 | Dexibuprofen | At least 30 |
| 2 | Diluent | 0 to 15 |
| 3 | Binder | 1 to 20 |
| 4 | Glidant | 0.1 to 10 |
| 5 | Super disintegrant | 0.1 to 15 |
| 6 | Optionally surfactant | 0 to 5 |

Compression aid

| | | |
|---|---|---|
| 7 | Diluent | 1 to 40 |
| 8 | Super disintegrant | 0.1 to 10 |
| 9 | Glidant | 0.1 to 5 |
| 10 | Lubricant | 0.1 to 7 |
| 11 | Optionally surfactant | 0 to 5 |

General manufacturing process of dexibuprofen ready to compress granules is as follows:

According to the present invention the novel process of preparing dexibuprofen granules which can be directly compressed in to tablets mainly consists of two steps: (a) preparation of base granule, and (b) blending or mixing of base granules with compression aid, wherein the process of preparing base granules, comprising the steps of: (a) mixing or blending of sifted dexibuprofen, and other pharmaceutically acceptable excipient(s), in fluid bed granulator, (b) granulation of fluidized dry mix obtained in step (a) using aqueous binder solution with top spray method to get wet granules, (c) drying of fluidized wet granules at suitable product bed temperature to obtain granules with loss on drying not more than 0.3% w/w at 50° C., (d) sizing the dried granules of step (c) using suitable method to get base granules, and (e) the blending of base granules with compression aid involves mixing or blending of base granules with previously sifted super disintegrant(s), glidant(s), lubricant(s) and diluent(s).

According to the another set of present invention the novel process of preparing dexibuprofen granules which can be directly compressed into tablets mainly comprise of two steps: (a) preparation of base granule, and (b) blending of base granules with compression aid, wherein the process of preparing base granules, comprising the steps of: (a) Slugging dexibuprofen through Roll compactor (b) Passing the obtained slugs through the multi mill fitted with 3.5 mm screen (c) The 3.5 mm screened granules were passed through #24 mesh and the retains passed through multi mill fitted with 1.5 mm screen (d) The 1.5 mm screened granules were again passed through #24 mesh. The #24 mesh passed granules of step c & d were passed through #40 mesh (e) the #24 mesh retains of step c & #40 mesh passed granules of step d were passed through roll compactor, and (f) the slugs obtained in step-e were repeated as per the steps from b to e. until a desired yield of #40 mesh retained granules are obtained (g) the blending of base granules with compression aid involves mixing or blinding of obtained base granules (f) with previously sifted super disintegrant(s), glidant(s), lubricant(s) and diluent(s).

According to the present invention following sets of experiment were performed to evaluate the effect of formulation process on compressibility and flowability of dexibuprofen granules.

In the first set of experiment wet granulation is performed using conventional rapid mixer granulator as described in example 1 and

In the second set of experiment wet granulation is performed using fluid bed granulator with top spray method as described in example (ex) 2, to 6 and.

In the third set of experiment, dry granulation is performed using slugging by roll compaction process with top spray method as described in example 7

Following examples are illustrative but no way limits the scope of the invention.

TABLE 2
Composition of dexibuprofen ready to compress granules:

| | | Ex 1 | Ex 2 | Ex 3 | Ex 4 | Ex 5 | Ex 6 |
|---|---|---|---|---|---|---|---|
| S. No | Ingredients | Quantity in mg | | | | | |
| | Base granulation | | | | | | |
| 1. | Dexibuprofen (250-350μ | 400 | 400 | 300 | 200 | 150 | 100 |
| | mean particle size) | | | | | | |
| 2. | Colloidal silicon dioxide | 5 | 5 | 3.75 | 2.5 | 1.88 | 1.25 |
| 3. | Croscarmellose sodium | 5 | 5 | 3.75 | 2.5 | 1.88 | 1.25 |
| 4. | Maize Starch (binder) | 61.5 | 61.5 | 46.13 | 30.75 | 23.06 | 15.375 |
| 5. | Lactose monohydrate | 61.5 | 61.5 | 46.1 | 30.75 | 23.06 | 15.375 |
| 6. | Purified water | qs. | qs. | qs. | qs. | qs. | qs. |
| | Granulation process | RMG | Fluidized bed processor granulator | | | | |
| | | granulator | | | | | |
| | Compression aid | | | | | | |
| 7. | Microcrystalline | 227 | 227 | 170.25 | 113.50 | 85.13 | 56.75 |
| | cellulose | | | | | | |
| 8. | Colloidal silicon dioxide | 10 | 10 | 7.5 | 5.0 | 3.75 | 2.5 |
| 9. | Sodium starch glycolate | 15 | 15 | 11.25 | 7.5 | 5.63 | 3.75 |
| 10. | Talc | 15 | 15 | 11.25 | 7.5 | 5.63 | 3.75 |
| | Film coating | | | | | | |
| 11. | Opadry II White | 24 | 24 | 18 | 12 | 9 | 6 |
| 12. | Purified water | qs. | qs. | qs. | qs. | qs. | qs. |

### Manufacturing Process:

### Example 1

### Dexibuprofen Ready to Compress Granules Prepared Using Rapid Mixer Granulator

1. Sift dexibuprofen through #30 mesh sieve, colloidal silicon dioxide and croscarmellose sodium through #60 mesh sieve, load and mix in rapid mixer granulator.

2. Preparation binder solution: disperse maize starch sufficient quantity of purified water to get uniform slurry and separately add lactose in sufficient quantity of purified water, then add starch slurry into boiling water under stirring to get thick paste. Allow the temperature of the paste to decrease to 50° C. and then add lactose slurry into paste and mix well.

3. Granulate the dry blend of step 1 using the binder solution prepared in step 2.

4. Dry the wet granules obtained in step 3 using fluid bed dryer (Glatt GPCG 1.1) at product bed temperature 25° C. to 30° C., to get dried granules with loss on drying not more than 0.3% w/w at 50° C.

5. Size the dry granule of step 4 through #24 mesh sieve and pass the retains through 1.0 mm screen using a cone mill.

6. Mix the sized dry granule of step 5 with previously sifted (#60 mesh sieve) blend of half the quantity of microcrystalline cellulose and colloidal silicon dioxide in blender for 3 minutes at 30 rpm.

7. Further add previously sifted (#60 mesh sieve) blend of remaining quantity of microcrystalline cellulose and sodium starch glycolate to step 6 and mix for 5 minutes at 30 rpm.

8. Finally lubricate blend of step 7 using previously sifted (#60 mesh sieve) talc by mixing for 2 minutes at 30 rpm.

9. Compress the blend of step 8 into tablets and pack into blister packs.

### Example 2

### Dexibuprofen Ready to Compress Granules Prepared Using Fluid Bed Granulator

1. Sift dexibuprofen through #30 mesh sieve, colloidal silicon dioxide and croscarmellose sodium through #60 mesh sieve, load and mix in fluid bed granulator (Glatt GPCG 1.1).

2. Preparation binder solution: disperse maize starch sufficient quantity of purified water to get uniform slurry and separately add lactose in sufficient quantity of purified water, then add starch slurry into boiling water under stirring to get thick paste. Allow the temperature of the paste to decrease to 50° C. and then add lactose slurry into paste and mix well.

3. Granulate the fluidized dry blend of step 1 using the binder solution prepared in step 2 using top spray method.

4. Dry the fluidized wet granules obtained in step 3 at product bed temperature 25° C. to 30° C., to get dried granules with loss on drying not more than 0.3% w/w at 50° C.

5. Size the dry granule of step 4 through #24 mesh sieve and pass the retains through 1.0 mm screen using a cone mill.

6. Mix the sized dry granule of step 5 with previously sifted (#60 mesh sieve) blend of half quantity of microcrystalline cellulose and colloidal silicon dioxide in blender for 3 minutes at 30 rpm.

7. Further add previously sifted (#60 mesh sieve) blend of remaining quantity of microcrystalline cellulose and sodium starch glycolate to step 6 and mix for 5 minutes at 30 rpm.

8. Finally lubricate blend of step 7 using previously sifted (#60 mesh sieve) talc by mixing for 2 minutes at 30 rpm.

9. Compress the blend of step 8 into tablets and pack into blister packs.

### Example 3

Dexibuprofen ready to compress granules prepared using fluid bed granulation process as followed in example 2.

### Example 4

Dexibuprofen ready to compress granules prepared using fluid bed granulation process as followed in example 2 and 3.

### Example 5

Dexibuprofen ready to compress granules prepared using fluid bed granulation process as followed in example 2, 3 & 4

### Example 6

Dexibuprofen ready to compress granules prepared using fluid bed granulation process as followed in example 2, 3, 4 & 6

### Example 7

Dexibuprofen ready to compress granules prepared using roll compactor:

| | | |
|---|---|---|
| Sr. No. | Ingredients | % by weight |

Base granules

| | | |
|---|---|---|
| 1 | Dexibuprofen | 100% w/w |

### Manufacturing Process:

### Example 1

### Dexibuprofen Ready to Compress Granules Prepared Using Roller Compactor by Slugging Method

1. Dexibuprofen was slugged through roller compactor.

2. The slugs were passed through multimill fitted with 3.5 mm screen at slow medium speed.

3. The 3.5 mm screened granules were passed through #24 mesh. The retained granules are collected and passed through multimill fitted with 1.5 mm screen at slow medium speed

4. The 1.5 mm screened granules were again passed through #24 mesh. The retained granules were collected separately.

5. The #24 mesh passed granules obtained in step-3 and in step-4 were passed through #40 mesh. The #40 mesh passed and retained granules were collected separately.

6. The #24 mesh retained granules obtained from step-4 and the #40 mesh passed granules obtained in step-5 were passed through roller compactor

7. The slugs obtained in step-6 were repeated as per the procedure from Step-2 to step-6. until desired yield of #40 mesh retained granules are obtained.

The granules obtained was subjected to particle size analysis by Malven spectroscopy and the following are the results.

| | PSD by Malven spectroscopy (in μm) | | | | | | |
|---|---|---|---|---|---|---|---|
| | | | | Trial | Trial | Trial | Trial |
| Tier | Reference | Trial 2 | Trial 3 | 4 | 5 | 6 | 7 |
| d(0.1) | 297.678 | 231.321 | 193.073 | NP | NP | NP | NP |
| d(0.5 | 511.139 | 579.937 | 541.913 | NP | NP | NP | NP |
| d(0.9) | 837.771 | 1057.919 | 1014.541 | NP | NP | NP | NP |
| Mean | 541.510 | 618.628 | 581.179 | 600.7 | 675.6 | 636.6 | 637 |

The dexibuprofen ready to compress granules prepared according to examples 1 to 5 were evaluated for compressibility index, flowability (Hausner Ratio), suitability for compressed on high speed commercial scale tablet compression machine and stability study at 30° C./65°/ORH, 40° C./75% RH, and stress stability test at 50° C./90% RH for one month.

TABLE 3
Compressibility index and flowability (Hausner Ratio) of
dexibuprofen ready to compress granules is as follows:

| Examples | Compressibility index | Flowability (Hausner Ratio) |
|---|---|---|
| Examples 1 | 15.9% | 1.19 |
| Examples 2 to 6 | 3.9% | 1.11 |
| Examples 7 | 7.89% | 1.086 |

The compressibility index of granules prepared by using fluid bed granulation process was found to be excellent (3.9%) compared with compressibility index of granules prepared by conventional granulation process using rapid mixer granulator i.e. fair (15.9%) and the flowability (Hausner Ratio) of granules prepared by using fluid bed granulation was found to be excellent (1.11%) compared with flowability (Hausner Ratio) of granules prepared by conventional granulation process using rapid mixer granulator i.e. fair (1.19%).

Further the compressibility index of granules prepared using slugging by roll compactor was found to be excellent (7.89%), which will be further improved upon addition of other additives & compression aid. The flowability (Hausner Ratio) of granules prepared by using slugging by roll compactor was found to be similar (1.086%) when compared with flowability (Hausner Ratio) of granules prepared by fluid bed granulation (1.11%). This can be further improved upon addition of other additives & compression aid.

As compressibility and flowability of granules depends on surface characteristics of particles, it was surprisingly found that the granules prepared using fluid bed granulation process showed improved compressibility and flowability compared to granules prepared by conventional granulation process using rapid mixer granulator, which may be due to individual particles coating with binder achieved in fluid bed granulation process and hence the surface characteristics of particles get modified in better way compared to conventional rapid mixer granulations process where individual particles coating with binder is not achieved.

In addition to the above, the ready to compress granules prepared using slugging by roll compactor showed similar compressibility and flowability to the fluid bed granulation process.

The advantage of granules prepared using slugging by roll compactor process is that the API as such is transformed into ready to compress granules (by milling and sizing the slugs formed) without help of any additives or compression aid. Whereas compared to granules prepared using fluid bed granulation process comprises mixture or blend of base granules and compression aid.

As said above, the compressibility and flow characteristics of ready to compress granules prepared by slugging method can be further improved than that of granules prepared using fluid bed granulation process by addition of mixture or blend of additives and compression aids to the base granules externally in less concentrations.

As said before the granules prepared using fluid bed granulation process will yield tablets without sticking when compressed on high speed commercial scale tablet compression machine, when compared to the granules prepared by conventional granulation process using rapid mixer granulator, which showed signs of sticking from the beginning of compression.

Like above, the granules prepared using slugging by roll compactor process will yield tablets without sticking. Further as it is a ready to compress granules, the incorporation of extra additives like disintegrant(s), glidant(s), lubricant(s) will be lesser percentages. Due to this cost factor for the formulation is reduced by skipping time and cost consuming factors like granulation, milling and addition of additives during granules processing and after processing granules to process into a ready to compress granules.

The tablets prepared according to example 2, to 6 showed no significant changes with respect to dissolution, assay, water content and Related substances at 40° C./75% RH for six months, 30° C./75% RH for six months and 25° C./60% RH for six month. Further 12 months stability studies of 30° C./75% RH and 25° C./60% RH are under progress.

TABLE 4
Stability summary of Dexibuprofen Tablets at Accelerated (40° C./75% RH)

| | | | | | | |
|---|---|---|---|---|---|---|
| | | | 1^{st} | 2^{nd} | 3^{rd} | 6^{th} |
| Parameters | Tentative limits | Initial | month | month | month | month |

Dissolution; Medium: pH 7.2 buffer; Volume: 900 ml; Apparatus: Type II; RPM: 50

| | | | | | | |
|---|---|---|---|---|---|---|
| Max | NLT 80 (Q) | 102 | 101.1 | 103.9 | 99.1 | 102.1 |
| Min | in 60 min | 92 | 98.3 | 98.2 | 90.3 | 94.8 |
| Avg | | 96 | 100 | 102 | 97 | 98 |

Related substances

| | | | | | | |
|---|---|---|---|---|---|---|
| BPPA (Imp B) % w/w | NMT 0.20 | BDL | 0 | 0.02 | 0 | 0.01 |
| IBPPA (Imp J) | NMT 0.20 | BDL | 0.01 | 1.05 | 0.01 | 0.02 |
| % w/w | | | | | | |
| 4-IBAP (Imp C) | NMT 0.20 | BDL | 0 | 0 | 0.02 | 0.03 |
| % w/w | | | | | | |
| Ibu Methyl ester | NMT 0.20 | BDL | 0.02 | 0.01 | 0.02 | 0.01 |
| % w/w | | | | | | |
| High unknown imp | NMT 0.17 | BDL | 0.01 | 0.02 | 0.05 | 0.08 |
| % w/w | | | | | | |
| Total Imp % w/w | NMT 0.8 | BDL | 0.07 | 0.06 | 0.13 | 0.21 |
| Assay | NLT 95%, | 99.9 | 100 | 98.2 | 98.9 | 98.3 |
| | NMT 105% | | | | | |
| Water content | NMT 5% | 3.04 | 2.81 | 2.88 | 2.87 | 2.96 |

TABLE 5
Stability summary of Dexibuprofen Tablets at
Intermediate (30° C./75% RH)

| | | | | |
|---|---|---|---|---|
| | Tentative | | | |
| Parameters | limits | Initial | 3^{rd} month | 6^{th} month |

Dissolution; Medium: pH 7.2 buffer; Volume: 900 ml;
Apparatus: Type II; RPM: 50

| | | | | |
|---|---|---|---|---|
| Max | NLT 80 (Q) | 102 | 97 | 98.8 |
| Min | in 60 min | 92 | 91.1 | 95.9 |
| Avg | | 96 | 95 | 97 |

Related substances

| | | | | |
|---|---|---|---|---|
| BPPA (Imp B) % w/w | NMT 0.20 | BDL | 0 | 0.01 |
| IBPPA (Imp J) % w/w | NMT 0.20 | BDL | 0 | 0.01 |
| 4-IBAP (Imp C) % w/w | NMT 0.20 | BDL | 0.01 | 0.01 |
| Ibu Methyl ester % w/w | NMT 0.20 | BDL | 0.01 | 0.01 |
| High unknown imp | NMT 0.17 | BDL | 0.03 | 0.01 |
| % w/w | | | | |
| Total Imp % w/w | NMT 0.8 | BDL | 0.08 | 0.05 |
| Assay | NLT 95%, | 99.9 | 98.7 | 99.4 |
| | NMT 105% | | | |
| Water content | NMT 5% | 3.04 | 2.98 | 2.98 |

TABLE 6
Stability summary of Dexibuprofen Tablets at
Long-Term (25° C./60% RH)

| | | | | |
|---|---|---|---|---|
| Parameters | limit | Initial | 3^{rd} month | 6^{th} month |

Dissolution; Medium: pH 7.2 buffer; Volume: 900 ml;
Apparatus: Type II; RPM: 50

| | | | | |
|---|---|---|---|---|
| Max | NLT 80 (Q) | 102 | 99.6 | 100.4 |
| Min | in 60 min | 92 | 92.2 | 94.2 |
| Avg | | 96 | 96 | 98 |

Related substances

| | | | | |
|---|---|---|---|---|
| BPPA (Imp B) % w/w | NMT 0.20 | BDL | 0 | 0 |
| IBPPA (Imp J) % w/w | NMT 0.20 | BDL | 0 | 0.01 |
| 4-IBAP (Imp C) % w/w | NMT 0.20 | BDL | 0 | 0 |
| Ibu Methyl ester % w/w | NMT 0.20 | BDL | 0.01 | 0.01 |
| High unknown imp | NMT 0.17 | BDL | 0.02 | 0.01 |
| % w/w | | | | |
| Total Imp % w/w | NMT 0.8 | BDL | 0.04 | 0.04 |
| Assay | NLT 95%, | 99.9 | 100.8 | 100.5 |
| | NMT 105% | | | |
| Water content | NMT 5% | 3.04 | 2.92 | 2.99 |

The preliminary stability study only with ready to compress granules, (without formulating the granules into tablets) prepared according to example 5 showed no changes with respect to appearance related substance and assay value,

As can be seen from Table 4 to 6 each of the tablets prepared in Examples 2 to 6 showed no significant changes with respect to dissolution, assay, water content and Related substances at 40° C./75% RH for six months, 30° C./75% RH for six months and 25° C./60% RH for six month.

Furthermore, a stress stability study was conducted at 50° C./90% RH for one month to check the possibility of tablet shape deformation due to low melting point of dexibuprofen. Tablet softening was observed in blister packs of tablets prepared according to example 1 (using rapid mixer granulator) leading to tablets shape deformation, but the blister packs of tablets prepared according to example 2 to 6 (using fluid bed granulator) showed no signs of tablet softening.

1. A process of preparing dexibuprofen ready to compress granules, comprising the steps of: (i) preparation of base granules using fluid bed granulation process, and (ii) mixing or blending of base granules with compression aid to get dexibuprofen ready to compress granules.

2. A process of preparing dexibuprofen ready to compress granules, comprising the steps of: (i) preparation of base granules using fluid bed granulation process with top spray method, and (ii) mixing or blending of base granules with compression aid to get dexibuprofen ready to compress granules.

3. A process of preparing dexibuprofen ready to compress granules, comprising the steps of: (i) preparation of base granules using dry slugging by roll compaction method and (ii) mixing or blending of base granules with additives and compression aid to get dexibuprofen pharmaceutical formulation

4. A process of preparing dexibuprofen ready to compress granules, comprising the steps of: (i) preparation of base granules using dry compaction by slugging in roll compactor and (ii) mixing or blending of base granules with additives and compression aid to directly compressed into tablets

5. A process of preparing dexibuprofen ready to compress granules, comprising the steps of: (i) preparation of base granules using fluid bed granulation process with top spray method, and (ii) mixing or blending of base granules with compression aid, wherein process of preparing base granules, comprising the steps of: (a) mixing or blending of sifted dexibuprofen, and other pharmaceutically acceptable excipient(s), in fluid bed granulator, (b) granulation of fluidized dry mix obtained in step (a) using binder solution with top spray method to get wet granules, (c) drying of fluidized wet granules at suitable product bed temperature, preferably between 25° C. to 30° C. to obtain granules with loss on drying not more than 0.3% w/w at 50° C., (d) sizing the dried granules of step (c) using suitable method to get base granules.

6. A process of preparing dexibuprofen granules which can be directly compressed into tablets mainly comprise of two steps: (a) preparation of base granule, and (b) blending of base grannies with additives/other pharmaceutically acceptable excipient(s) and compression aid, wherein the process of preparing base granules, comprising the steps of: (a) Slugging dexibuprofen through roll compactor (b) Passing the obtained slugs through the multi mill fitted with 3.5 mm screen (c) The 3.5 mm screened granules were passed through #24 mesh and the retains passed through multi mill fitted with 1.5 mm screen (d) The 1.5 mm screened granules were again passed through #24 mesh. The #24 mesh passed granules of step c & d were passed through #40 mesh (e) the #24 mesh retains of step c & #40 mesh passed granules of step d were passed through roll compactor, and (f) the slugs obtained in step-e were repeated as per the steps from b to e. until a desired yield of #40 mesh retained granules are obtained (g) the blending of base granules with compression aid involves mixing or blinding of obtained base granules (f) with previously sifted super disintegrant(s), glidant(s), lubricant(s) and diluent(s).

7. A process of preparing dexibuprofen ready to compress granules according to claim 5, wherein the sizing dried granules can be done using suitable size sieve and/or multi mill or cone mill.

8. A process of preparing dexibuprofen ready to compress granules according to claim 5, wherein the sizing the granules can be done using suitable size sieve/screen and for multi mill or cone mill, preferably with Multimill operating from slow speed to high speed, preferably at slow medium speed.

9. A process of preparing dexibuprofen ready to compress granules according to claim 1, wherein the compression aid may be comprise of one or more selected from the group of lubricant(s), glidant(s), super disintegrant(s), surfactant(s) and diluent(s).

10. A process of preparing dexibuprofen ready to compress granules according to claim 5, wherein the other pharmaceutically acceptable excipients may be comprise of one or more selected from the group of glidant(s), super disintegrant(s), diluent(s) and surfactant(s).

11. A process of preparing dexibuprofen ready to compress granules according to claim 6, wherein the sizing dried granules can be done using suitable size sieve and/or multi mill or cone mill.

12. A process of preparing dexibuprofen ready to compress granules according to claim 6, wherein the other pharmaceutically acceptable excipients may be comprise of one or more selected from the group of glidant(s), super disintegrant(s), diluent(s) and surfactant(s).

13. A pharmaceutical, free flowing particulate Dexibuprofen-containing composition comprising, by weight, at least 30% of Dexibuprofen; about 0 to about 15% of diluent; about 1 to about 20% binder; about 0.1 to about 10% of glidant about 0.1 to about 15% of super disintegrant; optionally about 0 to about 5% of surfactant, said composition having been prepared comprising the steps of: (i) preparation of base granules using fluid bed granulation process and (ii) mixing or blending of base granules with compression aid to get dexibuprofen pharmaceutical formulation, wherein process of preparing base granules, comprising the steps of: (a) mixing or blending of sifted dexibuprofen, and the said excipient(s), in fluid bed granulator, (b) granulation of fluidized dry mix obtained in step (a) using binder solution with top spray method or bottom spray method to get wet granules, (c) drying of fluidized wet granules at suitable product bed temperature, preferably between 25° C. to 30° C. to obtain granules with loss on drying not more than 0.3% w/w at 50° C., (d) sizing the dried granules of step (c) using suitable method to get base granules.

14. The composition of claim 13, wherein the compression aid is selected from the group consisting of diluent, super disintegrant, glidant, lubricant, surfactant and mixtures thereof.

15. The composition of claim 14, wherein the diluent is microcrystalline cellulose and comprises about 1 to 40% based on the weight of the composition.

16. The composition of claim 14, wherein the super disintegrant is sodium starch glycolate and comprises about 0.1 to 10% based on the weight of the composition.

17. The composition of claim 14, wherein the glidant is colloidal silicon dioxide and comprises about 0.1 to 5% based on the weight of the composition.

18. The composition of claim 14, wherein the lubricant is talc and comprises about 0.1 to 7% based on the weight of the composition.
